(19) United States
(12) Patent Application Publication
(10) Pub. No.: US 2008/0008762 A1
(43) Pub. Date: Jan. 10, 2008

(54) Steroid Formulation And Methods Of Treatment Using Same

(75) Inventors: Michael R. Robinson, Kensington, MD (US); George Grimes, Rockville, MD (US); Luisa V. Gravlin, Derwood, MD (US); Gopal K. Potti, Gaithersburg, MD (US); Peng Yuan, Rockville, MD (US); Karl G. Csaky, Kensington, MD (US)

Correspondence Address: EDWARDS ANGELL PALMER & DODGE LLP;(CLIENT REFERENCE NO. 47992), PO BOX 55874, BOSTON, MA, 02205, US

(73) Assignee: Government of the US, as represented by the Secretary, Department of Health and Human Services, Rockville, MD (US)

(21) Appl. No.: 11/791,125

(22) PCT Filed: Nov. 17, 2005
(86) PCT No.: PCT/US05/42332

§ 371 Date: May 17, 2007

Related U.S. Application Data
(60) Provisional application No. 60/628,741, filed on Nov. 17, 2004.

(51) Int. Cl. A61K 31/58; A61N 5/06; A61P 11/06; A61P 17/00; A61P 27/00; A61P 29/00; A61P 35/02; A61P 5/00; A61P 7/06

(52) U.S. Cl. 424/489; 514/169; 514/174; 607/88

(57) ABSTRACT

The invention provides steroid-containing pharmaceutical compositions which are free of classical preservatives and preferably comprise a steroid that is sparingly soluble or substantially insoluble in water, particulate steroid having an average particle size of from about 2.2 to about 10 microns. The pharmaceutical compositions can be used to treat medical conditions, including ophthalmological and back pain conditions.

### FIELD OF THE INVENTION

This invention pertains to pharmaceutical compositions comprising steroids, such as, but not limited to, triamcinolone acetonide. The invention further provides methods of administering steroid formulations of the invention to patients suffering from or susceptible to diseases and disorders which are routinely treated by steroid therapy.

### BACKGROUND OF THE INVENTION

Steroids that are soluble, sparingly soluble, and substantially-insoluble in water have many medical uses, and many formulations for administering steroids exist. Unfortunately, however, undesirable side effects accompany the administration of steroids to animals, including humans. Undesirable side effects often are more prevalent when the steroids are administered to sensitive tissues or systems such as the eye, musculoskeletal, dermatological, or cerebrospinal system.

A variety of preservatives have been used to maintain the sterility of steroid-containing pharmaceutical compositions. Similarly, a variety of dispersion agents have been used to control steroid suspendability of steroid compositions. Additionally, a variety of excipients have been used in steroid-containing pharmaceutical compositions. Steroid-containing pharmaceutical compositions also frequently comprise detergents, salts, buffers, and other additives generally in an effort to control the pharmacokinetics of the active ingredient and attenuate the severity and frequency of side-effects.

For example, intravitreal administration of triamcinolone acetonide has been widely used for the treatment of eye diseases, such as, but not limited to, diabetic retinopathy, uveitis, and choroidal neovascularization associated with age-related macular degeneration. The most commonly used formulation for intravitreal use is a triamcinolone acetonide formulation manufactured by Bristol-Myers Squibb (Princeton, N.J.) under the trademark Kenalog®. Unfortunately, the Kenalog® formulation administered intravitreally may cause sterile endophthalmitis and vision loss. Accordingly, there is a need in the art for other pharmaceutical compositions comprising steroids, especially for ocular, dermatological and musculoskeletal indications, which do not have the same profile of adverse events, and preferably are more effective and/or lessen undesirable side effects.

The invention provides such a pharmaceutical composition comprising a water-soluble, water-sparingly soluble, or water-insoluble steroid and methods of using the pharmaceutical composition. Embodiments of pharmaceutical compositions of the invention are is more effective than some prior art formulations and causes fewer side effects than other formulations. These and other advantages of the invention, as well as additional inventive features, will be apparent from the description of the invention provided herein.

### BRIEF SUMMARY OF THE INVENTION

The invention provides a pharmaceutical composition that comprises a glucocorticoid, angiostatic steroid, or other steroid and is free from classical preservatives. In preferred embodiments, the pharmaceutical composition is also free of dispersion agents and can consist of the steroid, a suitable excipient, a pharmaceutically acceptable salt, and water.

The invention also provides a pharmaceutical composition comprising a particulate steroid that is sparingly soluble or substantially-insoluble in water in which the steroid particles have an average particle size of from about 2.2 to about 10 microns. The pharmaceutical composition preferably comprises an excipient, but preferably does not contain preservatives or dispersion agents.

The excipient employed in the pharmaceutical composition is preferably selected from among methylcellulose, hydroxypropylmethylcellulose, hydroxyethylcellulose, and polyvinyl alcohol. More preferably, hydroxypropylmethylcellulose is used as the excipient in pharmaceutical compositions of the invention.

In a preferred embodiment, the pharmaceutical composition consists of (a) one or more active ingredients including at least one steroid that is sparingly soluble or substantially-insoluble in water having a steroid particle size of from about 2.2 to about 10 microns, (b) an excipient which is preferably selected from among a methylcellulose, an hydroxy-C₁-C₈ alkylmethylcellulose, Carbomer 940, polyethylene glycol, and polyvinyl alcohol, and is more preferably methylcellulose, hydroxypropylmethylcellulose, hydroxyethylcellulose, or polyvinyl alcohol, (c) a pharmaceutically-acceptable salt, and (d) water. Preferably, however, the pharmaceutical composition comprises only one active ingredient. The preferred active ingredient is triamcinolone acetonide.

In another preferred embodiment, the pharmaceutical composition is a preservative free triamcinolone acetonide pharmaceutical composition (also referred to as a TAC-PF pharmaceutical composition) which consists essentially of (a) a therapeutically effective amount of a particulate steroid which is sparingly soluble or substantially insoluble in water and has an average particle size of between about 2.2 microns (em) and about 10 microns, (b) an excipient selected from the group consisting of methylcellulose and hydroxy(C₁-C₈)alkylmethylcellulose, (c) an pharmaceutically acceptable salt, and (d) water, wherein the composition is substantially free of preservatives and non-polysaccharide polymers.

In another embodiment, the invention provides a pharmaceutical composition consisting essentially of: (a) particulate triamcinolone acetonide, (b) an excipient selected from the group consisting of methylcellulose and hydroxy(C₁-C₈)alkylmethylcellulose, (c) an pharmaceutically acceptable salt and (d) water, wherein the composition is substantially free of preservatives and non-polysaccharide polymers

The pharmaceutical compositions of the invention, including the TAC-PF pharmaceutical composition, can be used to treat an animal, which animal is preferably a human, in need of treatment with a steroid. Any condition amenable to treatment by steroids can be treated, but the inventive pharmaceutical composition is particularly well-suited to the treatment of tissues that can be sensitive to steroidal compositions such as tissues of the eye, skin, cerebrospinal or musculoskeletal system. The inventive pharmaceutical composition is particularly advantageous for periocular administration (including posterior juxtascleral, subconjunctival, anterior sub-Tenon's, posterior sub-Tenon's, retrobulbar and/or peribulbar administration), intravitreal, and transcleral administration. The inventive pharmaceutical composition is particularly advantageous for administration as an epidural, around the spine, intrathecally, interlaminar, through the intervertbral foramen, to a facet joint, to a disc, intraarticular, or intrabursal. In certain embodiments, the inventive pharmaceutical composition is suitable for transcleral administration.

### BRIEF DESCRIPTION OF THE DRAWINGS

FIG. 1 is a photograph image showing a white drug depot present in the vitreous eight (8) weeks after injection of a 16 mg dose of a preservative free triamcinolone acetonide formulation (TAC-PF) of Example 2.

FIGS. 2A and 2B are graphs that depict data from Example 1 indicative of the amount of steroid (triamcinolone acetonide) extracted from the vitreous of eyes injected with either 4 mg (FIG. 2A) or 16 mg (FIG. 2B) of the inventive steroid-containing pharmaceutical composition at the time points indicated by data points.

FIG. 3 is a plot of the estimated residual amount of drug in the vitreous following a 1-mg and 8-mg intravitreal injection of TAC-PF.

FIG. 4 is a plot of the relationship between the amount of TAC-PF injected into the vitreous of a mammal and the excretion half-life of the intravitreally deposited TAC depot.

FIG. 5 is a plot of the amount of triamcinolone acetonide extracted from the vitreous of a rabbit at various time points following a 4-mg Kenalog intravitreal injection and a regression line calculated from data.

FIG. 6 is a plot of serial ERG a- and b-wave amplitudes following a TAC-PF 16-mg intravitreal injection in the treated right eye of a rabbit.

FIG. 7A and 7B are photographs of representative histopathology section through the retina of a rabbit eye 20-weeks following an intravitreal injection of (A) 4-mg dose of TAC-PF, and (B) 4-mg dose of Kenalog®. Following the intravitreal Kenalog® injection, a loss of nuclei is apparent in the outer nuclear layer (red asterisk) and the arrows point to vacuolization of the outer segments. (original magnification 10×, hematoxylin and eosin stain).

FIG. 8A and 8B are photographs of an anterior subtenon's (ASTA) injection of a 20 mg dose of TAC-PF in a rabbit.

FIG. 9 is a bar graph of the amount of triamcinolone acetonide in the rabbit vitreous at days 0, 3, and 7 post anterior subtenon's injection (depicted in FIGS. 8A and 8B) for a 20 mg injection of TAC-PF (left bar) and 40 mg injection of Kenalog (right bar).

FIG. 10 is bar graph of triamcinolone acetonide concentration in the vitreous and aqueous of rabbits at day 0, 3, and 7 post injection of a 40 mg dose of TAC-PF into the anterior subtenon space measured by high pressure liquid chromatography.

FIG. 11 is bar graph of triamcinolone acetonide concentration in the vitreous and aqueous of rabbits at day 0, 3, and 7 post injection of a 20 mg dose of TAC-PF into the anterior subtenon space measured by high pressure liquid chromatography.

FIG. 12 is bar graph of triamcinolone acetonide concentration in the vitreous and aqueous of rabbits at day 0, 3, and 7 post injection of a 40 mg dose of TAC-PF into the posterior subtenon space measured by high pressure liquid chromatography.

FIG. 13 is bar graph of particle size distribution in a triamcinolone acetonide powder used in the formulation of TAC-PF pharmaceutical compositions of the invention.

### DETAILED DESCRIPTION OF THE INVENTION

The invention provides a pharmaceutical composition comprising a glucocorticoid, angiostatic steroid, or other steroid, which pharmaceutical composition preferably is free of preservatives, and which preferably also comprises particles of a steroid micronized to an average particle size of between about 2.2 and 10 microns, and more preferably consists of one or more active ingredients, an excipient, salt, and water. The steroid can be water-soluble, but preferably is sparingly soluble or substantially insoluble in water. The excipient is preferably polyvinyl alcohol, methylcellulose, a hydroxy-C₁-C₈ alkylmethylcellulose, or a hydroxy-C₁-C₈ alkylethylcellulose.

As the term is used herein, a “substantially water-insoluble steroid” refers to a particulate steroid that when suspended in 100 mL of deionized water at 25° C. as a powder having an average particle size of about 12 microns then less than about 10 mg of the steroid dissolves in the water.

As the term is used herein, a “sparingly soluble steroid” refers to a particulate steroid that when suspended in 100 mL of deionized water at 25° C. as a powder having an average particle size of about 12 microns then between about 10 mg and about 1 g of the steroid dissolves in the water.

Thus, in one embodiment, the invention provides a pharmaceutical composition comprising a glucocorticoid, wherein the pharmaceutical composition is free from classical preservatives. The pharmaceutical composition can be used to treat any suitable condition of an animal (e.g., human), including, but not limited to, conditions of the eyes, mucous membranes, and the musculoskeletal (including cerebrospinal) system. Tissues of the musculoskeletal system that can be suitably treated include all tissues in, and emanating from, the vertebral column, including but not limited to, the nerve roots and all peripheral nerves (e.g., the sciatic nerve and other peripheral nerves).

The pharmaceutical composition preferably comprises particles of a steroid, which are sparingly soluble or substantially insoluble in water. Steroid particles can have oblongate or irregular shapes. Accordingly, it is convenient and useful to define the size of the particle as the diameter of the smallest sphere that can encompass a particle. According to this definition, the particles preferably have a minimum average size of about 2.2 microns, more preferably about 2.5 microns, more preferably about 3 microns, and optionally about 4 microns. The particles also preferably have a maximum average particle size of about 10 microns, more preferably about 8 microns, yet more preferably about 7 microns, and most preferably about 5 microns. Moreover, the steroid particles preferably have a monophasic distribution.

Suitable glucocorticoids that can be employed include, but are not limited to, dexamethasone, fluoromethalone, medrysone, betamethasone, triamcinolone, triamcinolone acetonide, prednisone, prednisolone, hydrocortisone, rimexolone. Further examples include prednicarbate, deflazacort, halomethasone, tixocortol, prednylidene, prednival, paramethasone, methylprednisone, meprednisone, mazipredone, isoflupredone, halopredone acetate, halcinonide, formocortal, flurandrenolide, fluprednisone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone, desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, aldlometasone, 21-acetoxypregnenolone, tralonide, diflorasone acetate, deacylcortivazol, RU-26988, and deacyulcortivazol oxetanone. Triamcinolone acetonide, prednisolone, prednisolone acetate, rimexolone, flurormethalone, and fluromethalone acetate are preferred glucocorticoids. The steroid can also be a pharmaceutically acceptable salt of any of the foregoing, in which case the steroid salt is preferably insoluble, or more preferably sparingly soluble, in water.

The steroid can also be a hydrocortisoid.

Any suitable angiostatic steroid can be used, and is preferably selected from among hydrocortisone, tetrahydrocortisol-S, 11α-epihydrocotisol, cortexolone, 17α-hydroxyprogesterone, corticosterone, desoxycorticosterone, testosterone, estrone, dexamethasone, triamcinolone, and 6α-fluoro- 17,21-dihydroxy-16β-methyl-pregna-4,9,(11)-diene-3,20-dione. More preferably, the steroid is anecortave acetate.

The pharmaceutical composition preferably comprises a steroid that is sparingly soluble or substantially insoluble in water. Any suitable particulate water-insoluble or sparingly-soluble steroid can be used, however, the steroid preferably is a triamcinolone ((11β, 16α)-9-fluoro-11,17,18,21-dihydroxy-pregna-1,4-diene-3,20-dione) or one of its derivatives such as, but not limited to, triamcinolone diacetate (10, 16α)-16,21bis(acetyloxy)-9-fluoro-11,17-dihydroxypregna-1,4-diene-3,20-dione); triamcinolone hexacetonide ((11β,16α)-21-(3,3dimethyl-1-oxobutoxy)-9-fluoro-11-hydroxy-dihydroxy-16,17-[1-methyldethylidenebis(oxy)]-pregna-1,4-diene-3,20-dione), or triamcinolone betonide ((11β,16α)-21-[3-benzoylamino-2methyl-1-oxypropoxyl-9-fluoro-11-hydroxy-16,17-[1-methyldethylidenebis(oxy)]-pregna-1,4-diene-3,20-dione). Even more preferably, the triamcinolone derivative is triamcinolone acetonide ((11β,16α)-9-fluoro-11,21-dihydroxy-16,17-[1-methyldethylidenebis(oxy)]-pregna-1,4-diene-3,20-dione)).

The formulations are purified, non-preserved glucocorticoid formulations, and can be administered by any suitable route including those routes discussed herein.

The invention also provides a pharmaceutical composition consisting of a therapeutically effective amount of a particulate, water-insoluble or sparingly soluble steroid, an excipient, and an aqueous carrier. The inventive pharmaceutical composition can be used for any suitable purpose, but is particularly well-suited to ocular applications, especially intravitreal or periocular applications including posterior juxtascleral, anterior sub-Tenon's, posterior sub-Tenon's, or subconjunctival injections, as well as retrobulbar and/or peribulbar injections. Periocular applications may allow transcleral delivery of the pharmaceutical composition without injection directly into the vitreous. Periocular applications are preferred in treating indications at the posterior portion of the eye. For sub-Tenon's injections to treat indications at the posterior portion of the eye, the injection may be anterior sub-Tenon's because this route of administration can allow the attainment of higher steroid concentrations in the posterior segment of the eye.

The particulate steroid employed in the pharmaceutical composition can be of any suitable form. For example, the steroid can be in an amorphous form, semi-crystalline form, semi-amorphous form, or a mixture thereof. The steroid also can include one or more crystalline forms, and is preferably substantially in a crystalline form so that less than about 10% of the steroid particles are amorphous particles.

The inventive pharmaceutical compositions optionally comprise steroid particles having a controlled range of sizes. For example, less than about 20%, more preferably less than about 10%, yet more preferably less than about 5% or less than about 3% have a particle size of greater than 10 microns. The pharmaceutical composition is preferably substantially free of, or free of, steroid particles having a particle size of less than about 0.5 microns. Similarly, the pharmaceutical composition is preferably substantially free of, or free of steroid particles having a size of about 12 microns or greater, or more preferably about 10 microns or greater.

Any suitable method can be used to control the size of the steroid particles prior to incorporation into the pharmaceutical composition. Among the preferred methods of sizing the steroid particles is control of the manufacturing process and/or passing milled steroid particles through sizing sieves one or more times such that steroid particles that are too large or too small are excluded from the portion of the steroid incorporated into the pharmaceutical composition.

The pharmaceutical composition desirably comprises an excipient which allows the steroid particles to be suspended, and preferably remain suspended for a suitable time, upon mixing or agitation. Advantageously, substantially all of the steroid can be suspended in the pharmaceutical composition by vigorous shaking. Moreover, the steroid preferably remains substantially entirely suspended in the pharmaceutical composition for at least about 60 seconds, more preferably at least about 120 seconds, yet more preferably at least about 5 minutes, and even more preferably for at least about 10 or 15 minutes. More desirably, when the steroid is injected into the vitreous of the eye, the steroid rapidly aggregates and settles onto the floor of the vitreal space. For this reason, and to avoid the steroid settling on the retinal surface, it is desirable to maintain the injected animal in an upright position for at least about 15 minutes, more preferably about 30 minutes, and optionally about 2 hours following intravitreal administration of the inventive pharmaceutical composition. Preferably, a large enough proportion of the steroid settles to the floor of the vitreal space such that the animal's (preferably a human's) vision is not perceptibly impeded by the steroid about 24 hours, more preferably about 8 hours, even more preferably about 2 hours, and most preferably about 0.5 hours after intravitreal administration of the inventive pharmaceutical composition when the animal (or human) is maintained in an upright position for at least two hours after intravitreal administration.

Any animal can be treated. For example, rabbits, horses, dogs, cows, elephants, birds, mice, rats, and cats can be treated. Preferably, the animal is a human.

The excipient can be any suitable excipient. The excipient is selected from methylcellulose, hydroxy(C₁-C₈alkyl)methylcellulose, hydroxyethylcellulose, Carbomer 940, polyethylene glycol, or polyvinyl alcohol. In certain preferred formulations, including those pharmaceutical formulations consisting essentially of particulate triamcinolone acetonide, and an aqueous pharmaceutically acceptable salt, the excipient is selected from methylcellulose and hydroxy(C₁-C₈)alkylmethylcellulose.

In those formulations comprising polyvinyl alcohol, the polyvinyl alcohol is preferably made by the polymerization of vinylacetate monomer and is substantially hydrolyzed to polyvinyl alcohol. When the excipient is polyvinyl alcohol it preferably comprises less than about 20% polyvinylacetate, more preferably less than about 10% polyvinylacetate, even more preferably less than about 5% polyvinylacetate, and most preferably less than about 2% polyvinylacetate. That is, high hydrolysis grades of polyvinyl alcohol (ranging from <98% to over 99% hydrolysis) are preferred, and superhydrolysis grades of polyvinyl alcohol (having over 99% hydrolysis of polyvinylacetate) are more preferred. While not desiring to be bound by any particular theory it is believed that the highly hydrophobic character of high hydrolysis grade polyvinyl alcohol helps keep the steroid (e.g., triamcinolone acetonide) in depot form when administered to the vitreous cavity, or the like.

More preferably, the excipient is a methylcellulose. Yet even more preferably, the excipient is hydroxy-C₁-C₈ alkylmethylcellulose. Hydroxypropylmethylcellulose (HPMC or hypromellose) or hydroxyethylcellulose (HEC) are preferred hydroxy(C₁-C₈)alkylmethylcelluloses. The methylcellulose, HPMC, and HEC can be of any suitable type, e.g., pharmaceutical grade. HPMC is well known in the art.

HPMC can have varying degrees of methoxyl content and hydroxypropyl content. Common grades of HPMC have from about 15% to about 35% methoxyl content and from about 4% to 12% hydroxypropyl content. Preferably, however, the HPMC comprises from about 25% to about 35% methoxyl content and from about 7% to about 12% hydroxypropyl content. HPMCs having between 28% to 30% methoxyl content are more preferred. A preparation of methylcellulose suitable for use in the context of the invention is available from Dow Chemical Company (Midland, Mich.) under the trademarks E4M Methocel®, and preferably E4M Methocel Premium®.

Additionally, the excipient preferably has a low molecular weight. For example, when the excipient is a polysaccharide such as a methylcellulose, including but not limited to a hydroxyalkylmethylcellulose, then the excipient preferably has an average molecular weight of about less than about 100,000 daltons, more preferably less than about 90,000 daltons, and optionally less than about 85,000 daltons, and can have an average molecular weight greater than about 20,000 daltons or an average molecular weight of about 50,000 daltons, about 70,000 daltons, or about 80,000 daltons. While not desiring to be bound by any particular theory, the inventors believe that excipients in this size range suitably increase the viscosity of the solution, which assists in (among other things) administration of controlled quantities of the steroid to the animal, and which also permits the excipient to be cleared from an eye without tending to cause unacceptable rises in ocular pressure (i.e., avoiding the induction of glaucoma). The excipient, such as HPMC, can also have any suitable apparent viscosity. For example, in some embodiments a viscosity of from about 3000 to about 5600 cP is preferred. In certain other embodiments, a high viscosity HPMC can be preferred such as that disclosed by U.S. Pat. No. 5,422,376.

Any suitable concentration of excipient can be included in the inventive pharmaceutical composition. However, the pharmaceutical composition preferably contains a minimum excipient concentration of at least about 0.2%, more preferably about 0.35%, and even more preferably about 0.5%, wherein the percentages are measured in weight per volume. Additionally, the pharmaceutical composition preferably contains a maximum excipient concentration of about 5%, more preferably about 2%, and even more preferably about 1% excipient, again wherein these percentages are measured in weight per volume. Again, while not desiring to be bound by any particular theory, the inventors have observed that this range of excipient concentrations aids in the administration of accurate quantities of steroids, helps achieve a greater incidence and/or magnitude of therapeutic effect, and diminishes the occurrence of adverse side effects, such as (without limitation) glaucoma.

The steroid and excipient are preferably carried by an aqueous carrier, which is preferably a combination of a salt and water. Any suitable salt can be employed; however, the salt should be acceptable for pharmaceutical use in the concentration employed and is more preferably suitable for ophthalmological use and/or musculoskeletal use in the concentration employed. The salt is preferably sodium chloride. The pharmaceutical composition preferably contains at least about 0.7% (w/v) sodium chloride and no more than about 1.1% (w/v) sodium chloride (e.g., about 0.8-1% (w/v)). More preferably, the pharmaceutical composition contains about 0.9% sodium chloride. Additionally, the salt concentration or excipient concentration or both are preferably adjusted, if necessary, to provide an osmolarity of from about 200 mOsm to about 400 mOsm.

The pharmaceutical compositions of the invention preferably are also free of classical preservatives, such as, but not limited to, ophthalmologically and/or pharmaceutically acceptable preservatives. Classical preservatives are well known to the skilled artisan and include p-hydroxybenzoic acid esters, benzyl alcohol, quaternary ammonium compounds (in particular the mixture of alkyl benzyl dimethyl ammonium compounds known generically as “benzalkonium chloride”), benzoxonium chloride, cetylpridinium chloride, benzethonium chloride, cetyltrimethyl amnmonium bromide, chlorhexidine, poly(hexamethylene biguanide), BUSAN 77, ONAMER M, MIRAPOL A15, IONENES A, POLYQUATERNIUM 11, POLYQUATERNIUM 7, BRADOSOL, POLYQUAT D-17-1742, 1-octane sulfonic acid; 9-octadecenoic acid (sulfonated), ciprofloxacin, dodecyl diphenyloxide-disulfonic acid, dodecyl benzene sulfonate, sodium salts of fatty acids or tall oil, naphthalene sulfonic acid, sodium salts of sulfonated oleic acid, organic mercurials (such as thimerosal (sodium ethylmercurithiosalicylate)), thimerfonate sodium (sodium p-ethylmercurithiophenylsulfonate), 2,3-dichloro-1,4-naphthoquinone, 3-methyl-4-chlorophenol, 8-hydroxyquinoline (and derivatives thereof), bis(hydroxyphenyl) alkanes, bisphenols, chlorobutanol, chloroxylenol, dichlorophen[2,2′-methylene-bis(4-chlorophenol)], ortho-alkyl derivatives of para-bromophenol, ortho-alkyl derivatives of para-chlorophenol, oxyquinoline, para-alkyl derivatives of ortho-chlorophenol, para-alkyl derivatives of ortho-bromophenol, pentachlorophenyl laurate, phenolic derivatives such as 2-phenylphenol, 2-benzyl-4-chlorophenol, 2-cyclopentyl-4-chlorophenol, 4-t-amylphenol, 4-t-butylphenol, 4-chloro-2-pentylphenol, 6-chloro-2-pentylphenol, phenoxy fatty acid polyester, phenoxyethanol, and phenylethyl alcohol. Of course, this list is merely exemplary and not exhaustive. More preferably, the pharmaceutical composition is free of all preservatives irrespective of whether the preservatives have been approved for use in pharmaceutical compositions by the U.S. Food and Drug Administration or its equivalent agencies in other countries. Other preservatives which preferably are not incorporated into the inventive pharmaceutical composition include any chemical that inhibits endotoxin or pyrogen accumulation. For example, bacteriostats, microcides, and agents that kill or inactivate viruses are preferably not incorporated into the inventive pharmaceutical composition.

Moreover, the pharmaceutical composition preferably comports with current US Food and Drug Administration guidelines for endotoxin (including pyrogen) limits. According to the following manuscript, “Validation of Limulus Amebocyte Lysate Test as an End-Product Endotoxin Test for Human and Animal Parenteral Drugs, Biological Products, and Medical Devices” (December 1987), the current endotoxin limit using the Limulus Amebocyte Lysate Test is 0.5 Endotoxin Units (EU)/ml.

Additionally the pharmaceutical composition preferably is provided in a single unit dose vial or preloaded syringe, such that essentially the entire contents of the package can be usefully delivered to an animal in need of steroid treatment. The unit dose vial preferably contains enough steroid to be therapeutically effective for a human, and the indication to be treated can be any suitable condition. In certain preferred applications, the single unit dose vial or preloaded syringe of the pharmaceutical composition of the invention is suitable for use in administering the composition to either the eye, the cerebrospinal system, or to the musculoskeletal system. The pharmaceutical composition may be administered in a total volume of about 10 μl to about 2 ml, preferably about 100 μl to about 1 ml. The dose may also have a total volume of about 50 μl or less. The dose may preferably have a total volume of about 10 μl, 15 μl, 20 μl, 25 μl, 30 μl, 35 μl, 40 μl, 45 μl, 50 μl, 55 μl, 60 μl, 65 μl, 70 μl, 75 μl, 80 μl, 85 μl, 90 μl, 95 μl, 100 μl, 200 μl, 300 μl, 400 μl, 500 μl, 600 μl, 700 μl, 800 μl, 900 μl, or 1 ml or intermediate dosages. The dose may have a total volume greater than 1 ml, such as 1.1 ml, 1.2 ml, 1.3 ml, 1.4 ml, 1.5 ml, 1.6 ml, 1.7 ml, 1.8 ml, 1.9 ml, 2 ml, or more than about 2 ml, as well as intermediate dosages. The pharmaceutical composition is preferably administered in a single injection or, alternatively, in multiple injections, wherein multiple unit doses may be administered to the patient at the discretion of the treating physician based on the patient's size, medical condition, or other relevant criteria in determining the appropriate dosage. Preferably, a patient will receive a single dose. In some cases, a patient may receive multiple doses in a single treatment. In other cases, a patient who received a single dose may subsequently require additional doses. One or more subsequent doses may be administered at an appropriate interval after the first dose, such as about 4, 5, 6, 7, 8, 9, 10, or 11 or 12 months after the first treatment, or about 1 year after the first treatment. Subsequent doses may also be administered more than 1 year after the first treatment. One or more subsequent doses may be administered less than about 4 months after the first treatment, such as 1, 2, 3, 4, 5, 6, 7, 8, 9, 10, 11, 12, 13, 14, 15, or 16 weeks after the first dose, including intermediate intervals. The interval between a first and subsequent administration(s), as well as whether one or more subsequent administrations would improve the patient's medical condition, will be determined by the treating physician based on the patient's medical condition. When administered to the eye, the amount of steroid contained in the unit dose vial is preferably suitable for intravitreal or periocular and/or transcleral delivery such as subconjunctival, juxtascleral, or sub-Tenon's delivery. When administered to the spine, the amount of steroid contained in the unit dose vial is preferably suitable for epidural administration, and more preferably is suitable for epidural administration in a quantity calculated to relieve acute or chronic pain.

In certain treatable non-occular conditions identified herein the method of administration may preferably be selected from intradermal, intramuscular, subcutaneous, intraarticular, intranasal, aerosol spray, oral, transrectal, topical, intravenous, and the like. One of ordinary skill in the pharmacological art will readily identify preferred administration routes for a specific condition or disorder.

Additionally, the pharmaceutical composition preferably does not comprise a dispersion agent, such as, for example, polysorbate 80, ethanol, sorbitan trioleate, and tyloxapol. Other dispersion agents are well known to the skilled artisan and include (but are not limited to) polyethylene glycol 20 sorbitan monolaurate (Polysorbate 20), polyethylene glycol 5 soya sterol, Steareth-20, Ceteareth-20, PPG-2 methyl glucose ether distearate, cetyl phosphate, potassium cetyl phosphate, diethanolamine cetyl phosphate, polysorbate 60, glyceryl stearate, PEG-100 stearate, polyoxyethylene 20 sorbitan trioleate (also known as Polysorbate 85), polyoxyethylene 4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, stearamidopropyl PG-dimonium chloride phosphate, behenamidopropyl PG dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl methyl ammonium bromide, dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, ditallowyl oxyethyl dimethyl ammonium chloride, coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, amidobetaines, amidosulfobetaines, oleyl betaine, and cocamidopropyl betaine. Of course, this list is merely exemplary and not exhaustive.

In some preferred embodiments, the pharmaceutical composition consists of only the explicitly described components except for the option of containing other steroids or active drug agents. For example, the pharmaceutical composition can consist of the steroid, the excipient, water, and a pharmaceutically-acceptable salt. Further, these preferred embodiments can be administered alone or in combination with another active drug agent. In some embodiments, the active drug agent can be included in the pharmaceutical composition.

Among these preferred embodiments are pharmaceutical compositions consisting of triamcinolone acetonide, a methylcellulose, sodium chloride, and water. The triamcinolone acetonide is preferably present in the pharmaceutical composition in a concentration of from about 10 mg/ml to about 450 mg/ml, and even more preferably present in the pharmaceutical composition in a concentration of from about 10 mg/ml to about 200 mg/ml. The pharmaceutical composition may have a concentration of triamcinolone acetonide of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, or 450 mg/ml and intermediate concentrations. For dermatological uses, triamcinolone acetonide is preferably present in the pharmaceutical composition in a concentration of from about 1 mg/ml to about 20 mg/ml, such as 1, 2, 3, 4, 5, 6, 7, 8, 9, 10, 11, 12, 13,14, 15, 16, 17, 18, 19, or 20 mg/ml as well as intermediate concentrations. While not desiring to be bound by any particular theory, it is believed that the lower dose of triamcinolone acetonide lowers the chance of dermal atrophy and skin depigmentation. However, the pharmaceutical composition may be used in dermatological uses in any of the concentrations described above at the discretion of the treating physician. Additionally, the methylcellulose preferably is hydroxypropylmethylcellulose (hypromellose or HPMC), which is described elsewhere herein in more detail), and the HPMC preferably is a low molecular weight HPMC as described elsewhere herein. The concentration of excipients and salts of these preferred embodiments are also described elsewhere herein.

The inventive pharmaceutical composition preferably is non-toxic to the eye, and suitable for both scleral and transcieral delivery. For example, the inventive pharmaceutical composition preferably induces photoreceptor toxicity in less than 1%, more preferably less than about 0.5%, and even more preferably in less than about 0.1%, of humans to which it is administered intravitreally. Similarly, the inventive pharmaceutical composition preferably induces endophthalmitis and vision loss in less than 1%, more preferably less than about 0.5%, and even more preferably in less than about 0.1%, of humans to which it is administered intravitreally. Similarly, when a sufficient quantity of the inventive pharmaceutical composition is administered intravitreally to a 2 kg to 3 kg New Zealand White Rabbit so as to deliver 16 mg of the steroid, the inventive pharmaceutical composition preferably does not show histopathology or indications of toxicity as measured by serial electroretinography (ERG). While not desiring to be bound by any particular theory, it is believed that the non-toxic nature of preferred embodiments of the invention are due in part from the absence of preservatives and likely also from the absence of dispersion agents found in prior art formulations.

The inventive pharmaceutical composition preferably has a pH of from 5 to 9, more preferably from 6.8 to 7.8.

In certain other embodiments, the pharmaceutical composition has an osmolarity of from about 200 mOsm to about 400 mOsm.

The pharmaceutical composition described above can be administered alone or in combination with another suitable therapeutic agent. Additional suitable agents include, without limitation, methotrexate, cyclosporin, or both.

The inventive pharmaceutical composition can be advantageously administered before or after photodynamic therapy using verteporfin or similar agents, for example, to increase the durability of choroidal neovascular closure or for other suitable conditions. The inventive pharmaceutical composition can further be used in conjunction with other methods of choroidal neovascularization closure, which may increase the durability of choroidal neovascularization closure. Such other methods may include, but are not limited to, anecortave acetate injections, pegaptanib sodium (EYE 001, MACUGEN) injections, and rhuFabV2 injections.

Particularly when the particulate steroid is triaincinolone or a derivative thereof (e.g., triamcinolone acetonide), suitable additional therapeutic agents include, but are not limited to, anecortave acetate (4,9(11)-pregnadien-17α,21-diol-3,20dione-21-acetate) and/or 4,9(11)-pregnadien-17α,21-diol-3,20dione) (Alcon), EYE 001 (Eyetech), rhuFabV2 (Genentech), LY333531 (Lilly), and Fluocinolone (Bausch & Lomb). Thus, in another embodiment, the invention provides a composition consisting of at least two therapeutic agents, wherein one therapeutic agent is a particulate, water-insoluble or sparingly soluble steroid, an excipient selected from the group consisting of methylcellulose, hydroxy-C₁-C₈ alkylmethylcellulose, hydroxy-C₁-C₈ alkylethylcellulose, Carbomer 940, polyethylene glycol, and polyvinyl alcohol, a pharmaceutically acceptable salt, and water.

The pharmaceutical composition preferably is prepared using mechanical mixing of the steroid in a solution of the excipient under aseptic conditions. The mixing can be manual or automatic, but preferably does not substantially alter the size of the steroid particles. Additionally, the steroid can be, but need not be heated during the preparation of the formulation.

In certain embodiments, the pharmaceutical composition consists essentially of: (a) a therapeutically effective amount of a particulate steroid (which steroid (i) has an average particle size of from about 2.2 microns to about 10 microns, and (ii) is sparingly soluble or substantially insoluble in water), (b) an excipient selected from the group consisting of methylcellulose and hydroxy(C₁-C₈)alkylmethylcellulose, (c) an pharmaceutically acceptable salt, and (d) water. Preferred pharmaceutical compositions are substantially free of preservatives and non-polysaccharide polymers. Certain particularly preferred pharmaceutical compositions contain components (a), (b), (c), and (d) without additional ingredients incorporated into the formulation.

In certain other embodiments, the pharmaceutical composition consists essentially of: (a) particulate triamcinolone acetonide, (b) an excipient selected from the group consisting of methylcellulose and hydroxy(C₁-C₈)alkylmethylcellulose, (c) an pharmaceutically acceptable salt, and (d) water. Preferred pharmaceutical compositions are substantially free of preservatives and non-polysaccharide polymers. Certain particularly preferred pharmaceutical compositions contain components (a), (b), (c), and (d) without additional ingredients incorporated into the formulation. These formulations are referred to herein as TAC-PF formulations.

In certain preferred TAC-PF formulations, the pharmaceutical composition consists essentially of (a) 1-25 mg of particulate triamcinolone acetonide per milliliter of composition, (b) methylcellulose or hydroxypropylmethylcellulose at a concentration of about 0.2% (w/v) to about 5% (w/v), (c) sodium chloride present at a concentration of 0.7% (w/v) to about 1.1 (w/v); and water, wherein the composition is substantially free of preservatives and non-polysaccharide polymeric materials.

In certain other preferred TAC-PF formulations, the pharmaceutical composition consists essentially of (a) 2-20 mg of particulate triamcinolone acetonide per milliliter of composition, (b) hydroxypropylmethylcellulose at a concentration of about 0.2% (w/v) to about 5% (w/v), (c) sodium chloride present at a concentration of 0.7% (w/v) to about 1.1 (w/v); and, (d) water, wherein the composition is substantially free of preservatives and non-polysaccharide polymeric materials.

Preferred TAC-PF pharmaceutical compositions further consist essentially of one or more additional therapeutic agents.

Certain preferred TAC-PF pharmaceutical compositions are free of preservatives. Certain other preferred TAC-PF pharmaceutical compositions are free of dispersion agents.

In certain embodiments, the TAC-PF pharmaceutical composition has a particulate triamcinolone acetonide is in amorphous form, crystalline form, semi-crystalline form, semi-amorphous form, or a mixture thereof. In certain compositions, the particulate triamcinolone acetonide is at least partially in crystalline form, and about 10% or less of the steroid is in amorphous form.

The TAC-PF pharmaceutical compositions comprise triamcinolone acetonide particles having a controlled range of sizes. For example, less than about 20%, more preferably less than about 10%, yet more preferably less than about 5% or less than about 3% have a particle size of greater than 10 microns. Certain pharmaceutical compositions of the invention are preferably substantially free of, or free of, triamcinolone acetonide particles having a particle size of less than about 0.5 microns. Certain other pharmaceutical compositions of the invention are preferably substantially free of, or free of triamcinolone acetonide particles having a size of about 12 microns or greater, or more preferably about 10 microns or greater. One preferred range of particle sizes for TAC-PF pharmaceutical compositions is shown in FIG. 13. Certain preferred TAC-PF pharmaceutical composition contain triamcinolone acetonide particles have an average particle size of between about 2.2 microns and about 10 microns, between about 2.2 microns and about 7 microns or between about 3 microns and about 5 microns.

Certain preferred TAC-PF pharmaceutical compositions are packaged in single dose containers, e.g., a single dose vial. Preferred packaged TAC-PF pharmaceutical compositions are packaged in a single dose vial and contain written instructions regarding the administration of the packaged formulation for one or more of the indications identified herein. Certain preferred tissues to which the TAC-PF pharmaceutical compositions are suitable for administration include, but are not limited to tissues of the eye, musculoskeletal, dermatological, or cerebrospinal system. Certain preferred musculoskeletal tissues include joints (wrist, elbow, shoulder, knee, ankle, and the like), neck, and back and preferred cerebrospinal tissues include the neck, back, spinal cord and nerves.

The invention also provides a method of treating an animal for a condition in need of therapy. Any suitable animal can be treated. Certain preferred animals include mammals, more particularly, preferred animals include domesticated mammals (i.e., companionship mammals and livestock animals), primates, and humans. For example, the animal can be a rabbit, horse, dog, cow, elephant, bird, mouse, rat, pig, or cat. The animal is preferably a human. The method includes administering a therapeutically effective amount of the pharmaceutical composition of the invention to the animal so as to improve the animal's clinical condition or provide temporary or permanent relief from one or more symptoms.

Any suitable condition can be treated. The inventive pharmaceutical compositions are particularly well suited to treatment of ocular conditions. Among the ocular conditions amenable to treatment by the inventive pharmaceutical composition are retinopathy (which can be, e.g., proliferative or non-proliferative and can be of diabetic or non-diabetic etiology, e.g., radiation retinopathy), uveitis (with or without macular edema, and including without limitation intermediate uveitis and posterior uveitis), a neovascularization disorder such as choroidal neovascularization (of any etiology including, but not limited to, histoplasmosis syndrome, idiopathic, myopic degeneration, trauma, choroidal rupture, angioid streaks), posterior segment neovascularization, or iris neovascularization, macular degeneration (which can be exudative on non-exudative, and can be age-related or non-age-related), macular edema (which can be of any etiology including diabetic macular edema), vein occlusion (whether central, branch, or otherwise; either with or without macular edema), ocular ischemic syndrome, orbital inflammatory diseases, surgically induced inflammation, thyroid-related orbital inflammatory disease, endophthalmitis, pain from a blind eye, hypotony, ocular vascular tumors (including, but not limited to, retinal angiomatosis, capillary hemangiomas, orbital hemangiomas, periocular hemangiomas, angiomas, von Hippel-Lindau hemangioblastoma (with or without optic disc and/or macular edema), serous retinal detachment, chronic retinal detachment, idiopathic parafoveal telangectasia, iridocyclitis, papillitis, retinal vasculitis, keratitis (including without limitation peripheral ulcerative keratitis), corneal transplant rejection, corneal melts, autoimmune diseases of the cornea and sclera, autoimmune-related eye and orbital diseases, chalazion, orbital pseudotumor, scleritis, and episcleritis.

The inventive pharmaceutical compositions can also be used to improve visualization of the vitreous to assist in surgical procedures, including but not limited to, pars plana vitrectomy, internal limiting membrane peeling, macula hole repair, and epiretinal membrane removal.

Other conditions also can be treated with the inventive pharmaceutical compositions. Examples include (without limitation) diseases of the skin or mucous membranes, which include but are not limited to the mouth, nasopharynx, respiratory tract, and gastrointestinal system. Diseases of the skin include dermatitis, eczema, insect bites, lesions, ulcers, hemangiomas, vascular skin tumors, keloids, psoriasis, hypertrophic scars, traumatic scars, autoimmune skin disease, alopecia areata and other autoimmune disease that leads to hair loss, discoid lupus, esophageal strictures, and subglottic stenosis.

The inventive pharmaceutical compositions can also be used to treat a suitable musculoskeletal disease. These include without limitation bursitis, synovitis, tendonitis, capsulitis, arthritis (including without limitation osteoarthritis, psoriatic arthritis, idiopathic arthritis, and rheumatoid arthritis), epicondylitis, and fasciitis.

Other treatable conditions include asthma, clinical inflammation, epicondylitis, endocrine disorders, lupus, rheumatic carditis, herpes zoster ophthalmicus, colitis, irritable bowel syndrome, ulcerative colitis, gastroenteritis, Crohn's disease, hemolytic anemia, leukemia, lymphoma, and rhinitis.

The pharmaceutical compositions can be administered by any suitable means. Methods of administration other than topical or via eye drops, however, are preferred. Among the preferred routes of administration are juxtascleral injection or subconjunctival injection. More preferably, the pharmaceutical composition is injected into the vitreal space, sub-Tenon's, or into other periocular sites for transcleral administration. Although not wishing to be bound by theory, administration of the pharmaceutical compositions to the sub-Tenon's space or other periocular sites is suitable for delivery of at least a portion of the steroid to the vitreous of the treated eye. Thus, administration of a pharmaceutical composition of the invention by injection periocularly results in transcleral delivery of a steroid (e.g., triamcinolone acetonide) to the vitreous of the eye. Certain preferred transcleral administration routes include, but are not limited to, sub-Tenon's injection, injection posterior sub-Tenon's, injection anterior sub-Tenon's, injection posterior juxtasclerally, injection subconjunctivally, injection peribulbar, or injection retrobulbar.

In certain treatable non-occular conditions identified herein the method of administration may preferably be selected from intradermal, intramuscular, subcutaneous, intraarticular, intranasal, aerosol spray, oral, transrectal, topical, intravenous, and the like. One of ordinary skill in the pharmacological art will readily identify preferred administration routes for a specific condition or disorder.

Pharmaceutical compositions for transcleral treatment of the eye preferably have steroid particles (e.g., triamcinolone acetonide) having an average particle size of between 2.2 and 10 microns. Injection of pharmaceutical compositions having particles having an average particle size of between 2.2 and 10 microns provides enhanced drug delivery across the sclera into the vitreous of the eye. In certain other applications, including administration of the pharmaceutical compositions of the invention to other tissues, other particle size ranges can enhance drug delivery, drug release rate, or transport of the drug to the tissue in need of therapy. Thus, the size of the steroid particles (e.g., the triamcinolone acetonide particles) can be increased or decreased to provide desirable tissue penetration, inter-tissue transport or dissolution properties to enhance the effectiveness of the treatment.

The inventive pharmaceutical compositions are also particularly well suited to the mitigation of pain, especially including chronic or acute joint pain, back pain and neck pain. Any suitable route of administration can be used to deliver the pharmaceutical composition. Suitable routes of administration include around the spine, intrathecal, interlaminar, through the intervertebral foramen (e.g., for a targeted nerve root approach or “selective epidural injection”), to a facet joint, or to a disc. More preferred routes of administration include epidural, intraarticular, and intrabursal. All the foregoing routes of administration are particularly well suited to the treatment of sciatica, sciatic nerve compression, sciatic nerve root compression, multiple sclerosis, rheumatoid arthritis, neurodegenerative disease, autoimmune central nervous system disease, spinal stenosis, spinal tumors (with or without edema), post-laminectomy pain syndrome, pain following discectomy, herniated discs, degenerative spine disease, nerve root compression, post-herpetic neuralgia, radiculopathy, and neuralgia. Any suitable quantity of steroid can be administered, and when administered to a human can be in the range of about 10 mg to about 160 mg per injection, and more preferably about 40 mg to 80 mg per injection, preferably with no more than one injection per day.

In another embodiment, the invention provides a method of treating a person suffering from retinal edema or non-proliferative diabetic retinopathy which comprises administering an effective amount of a formulation free of a classical preservative and comprising a glucocorticoid.

The following examples further illustrate the invention but, of course, should not be construed as in any way limiting its scope.

### EXAMPLE 1

This example demonstrates that, in contrast to a commercially available formulation, pharmaceutical compositions lacking preservatives and dispersion agents do not give toxic side effects.

Triamcinolone acetonide USP grade (Voight Global Distribution, LLC, Kansas City, Mo.) was prepared as a sterile 40 mg/ml or 160 mg/ml suspension in single use vials by the Clinical Center Pharmacy Department at the National Institutes of Health. The suspending medium was normal saline USP (B. Braun Medical Inc., Irvine, Calif.). Hydroxypropylmethylcellulose (HPMC) 0.5% USP grade (Dow Chemical Company, Midland, Mich.) and was added to increase the viscosity of the formulation and enable the drug particles to stay in suspension for a minimum of 20 minutes after shaking the vial. Kenalog® formulation, a triamcinolone acetonide composition comprising dispersion agents and preservatives was obtained from Bristol-Myers Squibb.

New Zealand White rabbits of either sex and weighing 2-3 kg (Covance Laboratories, Inc., Vienna, Va.) were used, and all procedures adhered to the guidelines from the Association for Research in Vision and Ophthalmology regarding the use of animals in ophthalmic and vision research. Animals were anesthetized with ketamine hydrochloride (Fort Dodge, Inc., Fort Dodge, Ind.; 35 mg/kg) IM and xylazine (Phoenix Scientific, Inc., St. Joseph, Mo.; 5 mg/kg) IM. Proparacaine 1% ophthalmic drops (Allergan America, Hormigueros, PR) were used topically on the eye. The pupils were dilated with 1 drop each of phenylephrine hydrochloride 2.5% (Akom, Inc., Decatur, Ill.) and tropicamide 1% (Alcon, Inc., Humacao, PR). A baseline eye examination including fiudoscopy with an indirect ophthalmoscope, and an intraocular pressure measurement was performed.

After adequate anesthesia and akinesia were obtained, a lid speculum was placed, and the right eye was injected 4 mm behind the surgical limbus in the superotemporal quadrant with 0.1 ml of either the inventive triamcinolone acetonide composition (4 mg or 16 mg) or Kenalog® (4 mg). An anterior chamber paracentesis was performed to reduce the intraocular pressure in all rabbits.

Rabbits that received 4 mg and 16 mg doses were euthanized periodically over 4- and 8-month periods, respectively. Euthanasia was performed with an intracardiac pentobarbital overdose (Beuthanasia-D Special, Schering-Plough Animal Health Corp., Kenilworth, N.J.), and the right eye was enucleated and immediately frozen at −70° C. for later drug extraction. The eyes were dissected while frozen, and the vitreous humor was isolated using conventional methods. The triamcinolone acetonide was extracted by placing the vitreous in HPLC grade acetonitrile (Fisher Scientific, Pittsburgh, Pa.) in sealed vials for 24 hours at room temperature, sonicated using a GEX 600 Ultrasonic processor (Daigger, Lincolnshire, Ill.) for 60 seconds, and stored in sealed vials for another 24 hours at room temperature. The samples were spun-down in a Centra C12 centrifuge (Thermo IEC, Needham Heights, Mass.) for 3 minutes at 3,500 rpm, and the supernatants were submitted for HPLC analysis.

The drug assays were performed using an Agilent HP 1100 HPLC system (Agilent Technologies, Palo Alto, Calif.) equipped with a G1329A autosampler, a G1315A diode array detector, a G1312A binary pump, and a Dell workstation which controlled the operation of HPLC and analyzed the data. A Beckman Ultrasphere C-18 column (5 μm, 4.6×250 mm) (Beckman Coulter, Inc., Fullerton, Calif.) was used for separation, and detection was set at 254 mn. The flow rate employed was 1.0 ml/min with a mobile phase of 60% of acetonitrile and 40% of water by volume. The retention time was 7.0 minutes and the detection limit was 10 ng/ml.

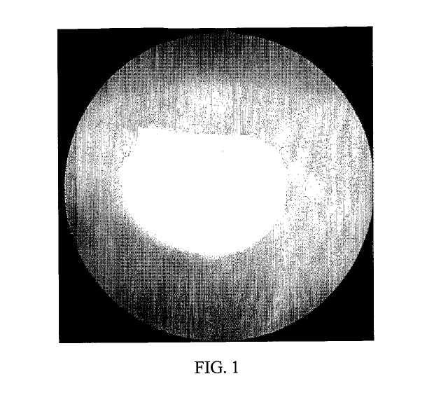

Triamcinolone acetonide (TA) particles that are injected into the vitreous aggregate to form an intravitreal depot. FIG. 1 is a photograph of the intravitreal depot formed after injection of TAC-PF intravitreally. On the assumption that the rate of TA elimination from the vitreous at any specific time depends on the remaining amount in the combined vitreous and depot, the experimental data were regressed with the following equation:
M=Mᵢ×exp(−kᵢ×t) (1)

t (day) time,

M (mg) represents the remaining amount of triamcinolone acetonide in the depot plus the vitreous,

Mᵢ (mg) represents the initial injected amount, and

kᵢ (day⁻¹) is the elimination rate constant that depends upon Mi. The elimination rate constants for the 4-mg injection and 16-mg injection, k₄ or k₁₆, were found by regressing Equation (1) to the animal experimental data from the 4-mg and 16-mg injections using a standard spreadsheet program.

The elimination rate constants were assumed to be related to the initial amounts by the following equation:
k₄/k₁₆=(M₄/M₁₆)ⁿ⁻¹ (2)
From the calculated k₄, k₁₆, and doses, M₄=4 mg and M₁₆=16 mg, a value for was determined. Equation (2) permitted estimation of the elimination for other intravitreal triamcinolone acetonide doses.

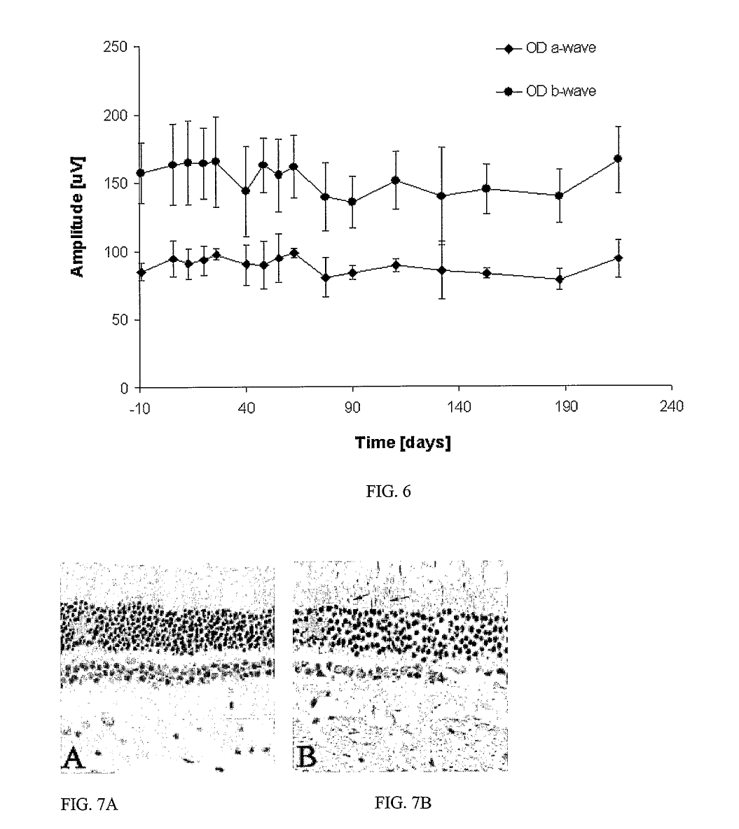

Ocular Toxicity: New Zealand White rabbits were anesthetized and injected in the right eye in the same manner as described above with 0.1 mL (4 mg or 16 mg) of the inventive pharmaceutical composition. Electroretinography (ERG) was performed at baseline (pre-injection) and then periodically over a 4-month and 7-month period, for the 4 mg and 16 mg dose, respectively. ERGs were recorded under anesthesia with dilated pupils from each eye separately after 30 minutes of dark adaptation. A monopolar contact lens electrode (ERG-jet, La Chaux des Fonds, Switzerland) was placed on the cornea and served as a positive electrode. Subdermal needle electrodes inserted in the forehead area and near the outer canthus served as the ground and negative electrodes, respectively. ERGs were elicited by flash stimuli delivered with a Grass PS22 photostimulator (Grass Instruments, Quincy, Mass.) at 0.33 Hz. Responses were amplified, filtered, and averaged with a Nicolet Spirit Signal averager (Nicolet Instruments Corps., Madison, Wis.). The mean of 20 responses was measured to obtain amplitude values of a-waves and b-waves (FIG. 6). Rabbits were euthanized, and both eyes were enucleated 2 weeks following the last ERG. Enucleated eyes were fixed in 10% formalin immediately after removal. Paraffin sections through the pupillary-optic nerve head axis including the injection sites were stained with hematoxylin and eosin for light microscopic examination.

Statistical Analysis: The mean of the ERG amplitudes for all rabbits at each time point was calculated and statistical analysis was performed separately on all right (treated) eyes and then on all the left (untreated) eyes. The differences in the mean ERG amplitudes at each recording from the baseline (pre-implant) values were compared and tested by the analysis of variance (ANOVA) using PSI-Plot version 7.0 (Poly software International, Inc., Pearl River, N.Y., USA). Differences were considered likely to be clinically significant if the P-value was <0.05.

TABLE 1

| | | | Drug depot |
|---|---|---|---|
| | | | present in |
| Intravitreal dose | Elimination rate | Half-life | the vitreous [days] |
| and formulation | constant (k) | [days] | (estimated) |
| 1-mg TAC-PF | 0.047 | 15 | 75 |
| 4-mg TAC-PF | 0.029 | 24 | 120 |
| 8-mg TAC-PF | 0.023 | 30 | 150 |
| 16-mg TAC-PF | 0.018 | 39 | 195 |
| 4-mg Kenalog ® | 0.030 | 23 | 115 |

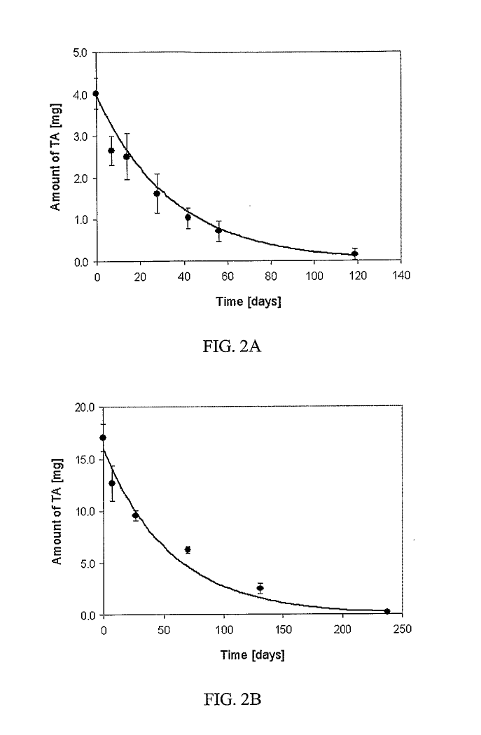

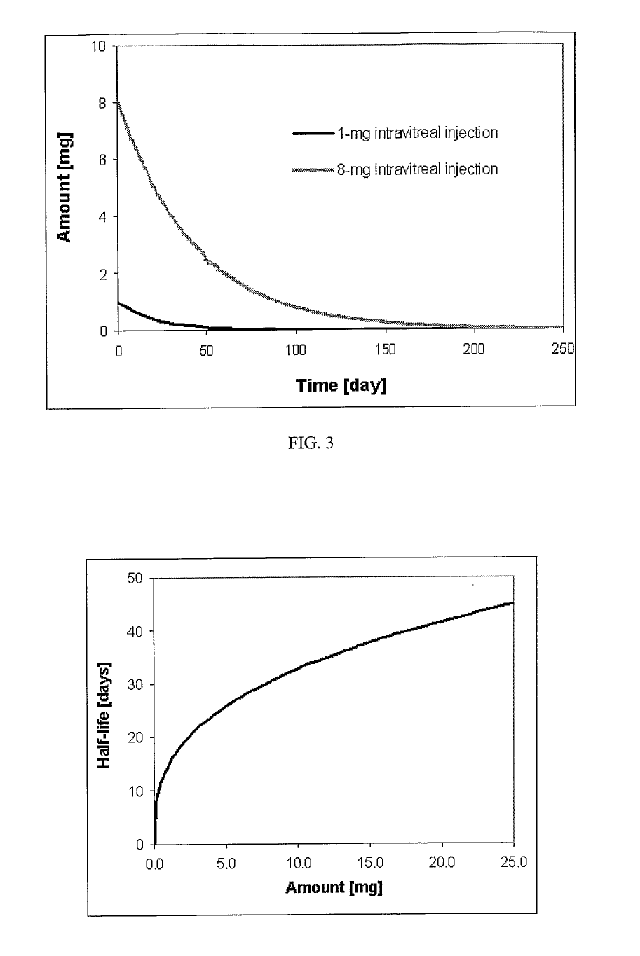

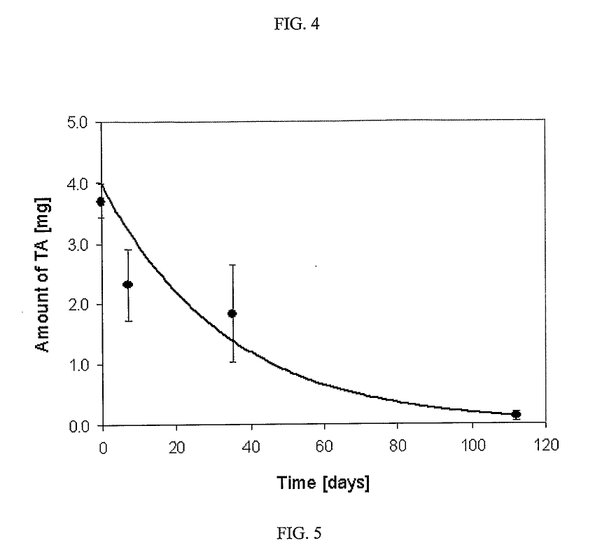

Ocular Pharmacokinetics Results: A total of 68 rabbits were injected with TAC-PF or Kenalog® and 4 rabbits were euthanized at each time point. There were no detectable levels of triamcinolone acetonide in the aqueous humor of all rabbits. In the TAC-PF groups, the amount of triamcinolone acetonide extracted from the vitreous at each time point is shown as dots in FIG. 2 (A: 4-mg TAC-PF intravitreal injection; B: 16-mg TAC-PF intravitreal injection). Both sets of data were regressed with Equation (1) and the results are shown as solid lines in FIGS. 2A and 2B. The elimination rate constants for the 4-mg (k₄) and 16-mg (k₁₆) TAC-PF injections were found to be 0.029 [day⁻¹] (R²=0.99) and 0.018 [day⁻¹] (R²=0.97), respectively. The relationship between the rate constants for the 4-mg and 16-mg TAC-PF injections give a value of n=0.66 from Equation (2). Equation (2) was generalized to the following form to predict the rate constant value, kᵢ in day⁻¹, for any injection amount, Mᵢ in mg,
kᵢ=0.047×Mᵢ^{-0.34} (3)
The rate constants for 1-mg and 8-mg TAC-PF was calculated to be k₁=0.047 and k₈=0.023 from Equation (1), respectively and FIG. 3 shows estimated residual amount of 1-mg and 8-mg injected TAC-PF in the vitreous. The half-life of each injected amount was calculated with the following Equation (4) and the relationship between the initial injected amount [mg] and the half-life is shown in FIG. 4.
Half-life [days]=0.693/kᵢ (4)

The experimental data of 4-mg Kenalog® intravitreal injection were analyzed using the same methods as with the TAC-PF injection and found to be k₄ (Kenalog®)=0.030 [day⁻¹] (R²=0.97) (FIG. 5). With an assumption that injected triamcinolone acetonide stays in the vitreous for five times the half-life, the duration of time the drug depot would be present in the vitreous was calculated (Table 1). A total of 68 rabbits were injected with the inventive pharmaceutical composition or the conventional Kenalog® foundation, and 4 rabbits were euthanized at each time point. There were no detectable levels of triamcinolone acetonide in the aqueous humor of all rabbits. In the groups treated with the inventive pharmaceutical composition, the amount of triamcinolone acetonide extracted from the vitreous at each time point is shown as dots in FIGS. 2A and 2B (reflecting, respectively, 4 mg and 16 mg intravitreal injections of the inventive pharmaceutical composition). Both sets of data were regressed with Equation (1), and the results are shown as solid lines in FIGS. 2A and 2B. The elimination rate constants for the 4 mg (k₄) and 16 mg (k₁₆) injections of the inventive composition were found to be 0.029 [day⁻¹] (R²=0.99) and 0.018 [day⁻¹] (R²=0.97), respectively. The relationship between the rate constants for the 4 mg and 16 mg injections of the inventive pharmaceutical composition gave a value of n=0.66 from Equation (2). Equation (2) was generalized to the following form to predict the rate constant value, kᵢ in day⁻¹, for any injection amount, Mᵢ in mg,
kᵢ=0.047×Mᵢ^{-0.34} (3)

The rate constants for 1 mg and 8 mg of the inventive pharmaceutical composition was calculated to be k₁=0.047 and k₈=0.023 from Equation (1), respectively. The half-life of each injected amount was calculated with the following Equation (4), and the relationship between the initial injected amount [mg] and the half-life is shown in FIG. 3.
Half-life [days]=0.693/kᵢ (4)

The experimental data of 4 mg Kenalog® formulation intravitreal injection were analyzed using the same methods as with the injection of the inventive pharmaceutical composition and found to be k_{4(Kenalog®)}=0.030 [day⁻¹] (R²=0.97). With an assumption that injected triamcinolone acetonide stays in the vitreous for five times the half-life, the duration of time the drug depot was determined to be about 120 days for 4 mg of the inventive pharmaceutical composition and about 115 days for 4 mg of Kenalog®.

Ocular Toxicity Results: A total of 19 rabbits received 4 mg of triamcinolone acetonide in the inventive pharmaceutical composition (n=9), 16 mg of triamcinolone acetonide in the inventive pharmaceutical composition (n=6), or a 4 mg of triamcinolone acetonide in the conventional Kenalog® formulation (n=4) by intravitreal injection. Rabbits are not consistent corticosteroid responders; however, monthly intraocular pressure measurements were performed with general anesthesia, and the results showed no increases over baseline in all groups. Clinical examination throughout the study period showed normal cornea, anterior chamber, lens, vitreous, and retina, in all 3 groups. The ERGs in the treated eyes and untreated eyes with both the 4 mg and 16 mg doses of the inventive pharmaceutical composition showed no significant changes in the a-wave or b-wave amplitudes during the study period. Histopathology on the rabbit eyes receiving a 4 mg dose of the inventive pharmaceutical composition showed normal tissues by light microscopy at 10 (n=4) and 20 (n=5) weeks. FIG. 7A is a photographic image of a representative eye receiving 4 mg of TAC-PR composition. Histopathology on the rabbit eyes receiving a 16 mg dose of inventive pharmaceutical composition was normal at 38-weeks (n=6). However, histopathology of rabbit eyes (n=4) receiving a 4 mg dose of the Kenalog® formulation at 20-weeks showed retinal toxicity in all eyes. FIG. 7B is a photographic image of a representative eye receiving 4 mg of Kenalog®. There was decreased nuclei density in the outer nuclear layer of the treated eye, vacuolization of the photoreceptors, shortening of the outer segments, and swelling of inner nuclear cells. These retinal changes were present in the region of the medullary rays and the peripheral retina in all sections. The histopathology of the remainder of the ocular tissues was normal.

Thus, the results of this example demonstrate that the half-life of 4 mg triamncinolone acetonide in the inventive pharmaceutical composition, 16 mg triamcinolone acetonide in the inventive pharmaceutical composition, and 4 mg of triamcinolone acetonide in the conventional Kenalog® formulation was about 24 days, 39 days, and 23 days, respectively. Moreover, while the 16 mg dose of triamcinolone acetonide in the inventive preservative-free formulation induced no histopathological toxicity, the 4 mg dose of the Kenalog® formulation did show retinal toxicity. These data suggest that higher concentrations of triamcinolone acetonide may be administered to the eye in accordance with the present invention without the toxicity resulting from lower-dose administration of conventional formulations containing triamcinolone acetonide.

### EXAMPLE 2

This example shows a preservative-free, dispersion agent-free composition of the invention.

| | | Required | |
|---|---|---|---|
| | Quantity | Batch | |
| Ingredient | Per Unit | Quantity | |
| Triamcinolone | 160 mg | 32 | g |
| Acetonide Powder | | | |
| Methocel E4M | 0.5% | 1 | g |
| (Hydroxypropyl | | | |
| Methylcellulose) | | | |
| Powder | | | |
| 0.9% NaCl | 1 ml | 200 | mL |
| injection USP QS | | | |
| to | | | |

### EXAMPLE 3

This example shows a preservative-free, dispersion agent-free composition of the invention.

| | | Required | |
|---|---|---|---|
| | Quantity | Batch | |
| Ingredient | Per Unit | Quantity | |
| Triamcinolone | 40 mg | 4.57 | g |
| Acetonide Powder | | | |
| Methocel E4M | 0.5% | 0.571 | g |
| (Hydroxypropyl | | | |
| Methylcellulose) | | | |
| Powder | | | |
| 0.9% NaCl | 1 ml | 109.68 | mL |
| injection USP QS | | | |
| to | | | |

### EXAMPLE 4

This example shows a preservative-free composition of the invention.

| | Quantity |
|---|---|
| Ingredient | Per Unit |
| Triamcinolone | 40 mg |
| Acetonide Powder | |
| Methocel E4M | 0.5% |
| (Hydroxypropyl | |
| Methylcellulose) | |
| Powder | |
| Polysorbate-80 | 0.05% |
| 0.9% NaCl injection | 1 ml |
| USP QS to | |

### EXAMPLE 5

This example shows that a patient suffering from macular edema experienced improvement in the condition after administration of a micronized, preservative-free, dispersion agent-free composition of the invention.

A patient with a greater than a 20-year history of insulin dependent diabetes mellitus developed severe macular edema and vision loss. To treat the macular edema that was refractory to standard photocoagulation, the patient had an intravitreal injection of the formulation of Example 2 performed in the left eye, and within 1 week, experienced a visual improvement in this eye. The patient has had a dramatic reduction in the fluorescein leakage in the macula and a large decrease in the central macular thickness on Optical Coherence Tomography (OCT) from 929 microns pre-injection to 241 microns 3 weeks later.

Accordingly, the results of this example demonstrate that the inventive pharmaceutical composition can be therapeutically administered to patients suffering from macular edema, vision loss, or both.

### EXAMPLE 6

This example shows that the composition of the invention provides superior treatment of the intravitreal portion of the eye when administered periocularly as compared to the conventional Kenalog® formulation.

The inventive pharmaceutical composition of Example 3 was administered in the subconjunctival space to provide 20 mg of triamcinolone acetonide. Separately, the Kenalog® formulation was administered in the subconjunctival space to provide 40 mg of triamcinolone acetonide. The concentration of triamcinolone acetonide was measured in the vitreous. For the composition of Example 3, about 7 μg of triamcinolone acetonide was found in the vitreous zero days after administration, about 1 μg of triamcinolone acetonide was found in the vitreous three days after administration, and about 1.5 μg of triamcinolone acetonide was found in the vitreous seven days after administration. For the Kenalog® formulation, about 0.5 μg or less of triamcinolone acetonide was found in the vitreous zero days after administration, about 1.5 μg of triamcinolone acetonide was found in the vitreous three days after administration, and about 1 μg of triamcinolone acetonide was found in the vitreous seven days after administration. Thus, the composition of Example 3 provided much higher initial concentrations of triamcinolone acetonide in the vitreous than a double-dose of the Kenalog® formulation and provided comparable concentrations of triamcinolone acetonide through the middle and end of the first week after administration.

Thus, the results of this example demonstrate that the inventive pharmaceutical composition has advantages over the use of conventional formulation for subconjunctival or periocular administration of triamcinolone acetonide to the vitreous.

### EXAMPLE 7

This example shows that the inventive pharmaceutical composition can be administered to successfully treat choroidal neovascularization with progressive vision loss.

An elderly man with a history of age-related macular degeneration developed choroidal neovascularization with progressive visual loss in his right eye. The patient had an intravitreal injection of the composition of Example 3 performed in the right eye and a follow-up fluorescein angiogram showed a significant decrease in leakage seen in the posterior pole documented 1 week after the injection. The patient had stabilization of his vision and no further Vision loss over the next 3 months.

### EXAMPLE 8

This example shows that the inventive pharmaceutical composition can be administered to successfully treat macular edema, particularly macular edema refractive to standard laser therapy.

A man with a history of type II diabetes of 10 years of duration, presented with vision loss in both eyes. Examination showed clinical significant macular edema which did not respond to standard laser therapy. He also developed submacular fibrosis with further deterioration of his vision. In addition, he developed severe nonproliferation diabetic retinopathy in both eyes. An intravitreal injection of the composition of Example 3 was performed in his left eye. He had an improvement in his vision after 1 week and a clear reduction in fluorescein leakage in the macula with angiography. There was a stabilization of the submacular fibrosis over a 4-month follow-up period.

### EXAMPLE 9

This example shows that the inventive pharmaceutical composition can be administered to successfully treat central retinal vein occlusion.

A man presented with an incomplete central retinal vein occlusion with macular edema. An intravitreal injection of the composition of Example 3 was performed and he had a dramatic improvement in venous perfusion and a reduction in macular edema that was documented over a 6-month period. After the triamcinolone acetonide disappeared from the vitreous cavity, the vein occlusion and macular edema recurred. A second intravitreal injection of the composition was performed, and again, he developed a significant improvement in venous perfusion and a reduction in macular edema that has been stable with a follow-up of 3 months.

### EXAMPLE 10

This example shows that triamcinolone acetonide administered by sub-Tenon's injection with subsequent localization of at least a portion of the triamcinolone acetonide from the sub-Tenon's depot to the aqueous and vitreous of the eye.

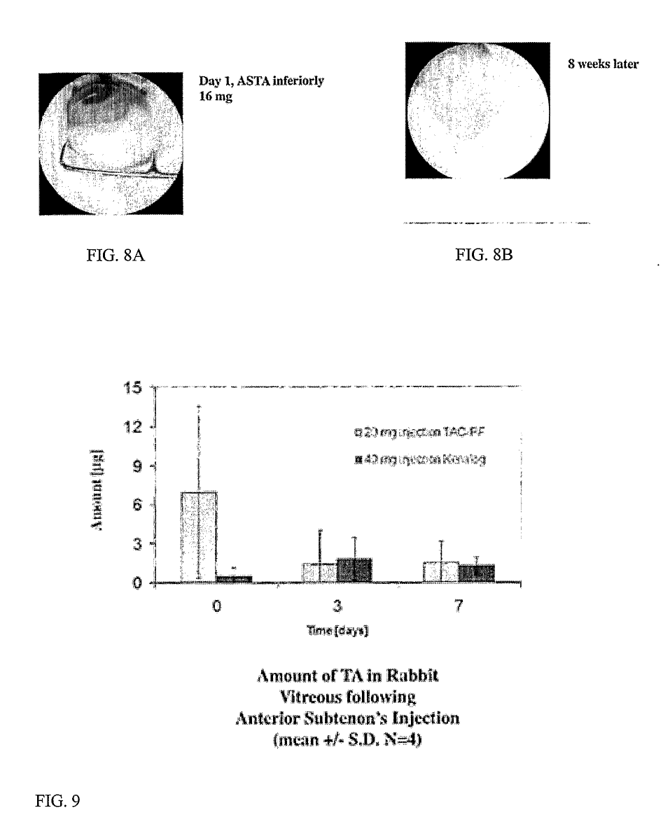

Prior to a sub-Tenon's injection or other ophthalmic surgical procedure, rabbits were anesthetized with ketamine hydrochloride (Fort Dodge, Inc., Fort Dodge, Ind.; 35mg/kg) IM and xylazine (Phoenix Scientific, Inc., St. Joseph, Mo.; 5mg/kg) IN; proparacaine 1% ophthalmic drops (Allergan America, Hormigueros, PR) were used topically on the eye. The pupils were dilated with 1 drop each of phenylephrine hydrochloride 2.5% (Akom, Inc., Decatur, Ill.) and tropicamide 1 % (Alcon, Inc., Humacao, PR). After adequate anesthesia and akinesia were obtained, a lid speculum was placed (FIG. 8A) and the right eye was injected with a triamcinolone acetonide preservative-free formulation. A sub-Tenon's injection was performed in the superotemporal quadrant of the right eye with the center of the depot 5-6 mm from the limbus using a 30 gauge needle (FIG. 8B). At various times points the animal was euthanized with an intracardiac pentobarbital overdose (Beuthanasia-D Special, Schering-Plough Animal Health Corp., Kenilworth, N.J.). The treated eye was enucleated and immediately frozen at −80° C. The eyes were dissected while frozen and the vitreous and aqeuous humor was isolated. The triamcinolone acetonide was extracted by placing the vitreous or aqueous in HPLC grade acetonitrile (Fisher Scientific, Pittsburgh, Pa.) in sealed vials for 24 hours at room temperature, sonicated using a GEX 600 Ultrasonic processor, (Daigger, Lincolnshire, Ill.) for 60 seconds, and stored in sealed vials for another 24 hours at room temperature. The samples were spun down in a Centra C12 centrifuge (Thermo IEC, Needham Heights, Mass.) for 3 minutes at 3,500 rpm and the supernatants were submitted for HPLC analysis. The drug assays were performed using an Agilent HP 1100 HPLC system (Agilent Technologies, Palo Alto, Calif.) equipped with a G1329A autosampler, a G1315A diode array detector, a G1312A binary pump, and a Dell workstation which controlled the operation of HPLC and analyzed the data. A Beckman Ultrasphere C-18 column (5 um, 4.6×250 mm)(Beckman Coulter, Inc., Fullerton, Calif.) was used for separation, and detection was set at 254 nm. The flow rate employed was 1.0 ml/min with a mobile phase of 60% of acetonitrile and 40% of water by volume. The retention time was 7.0 min and detection limit was 10 ng/ml.

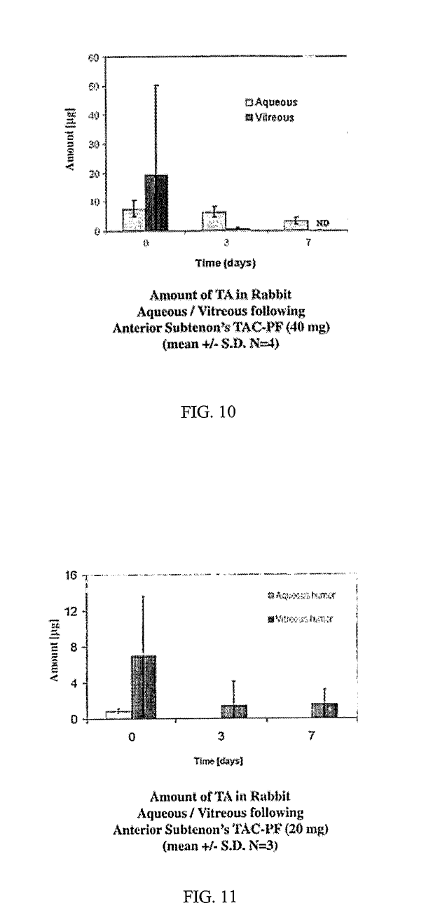

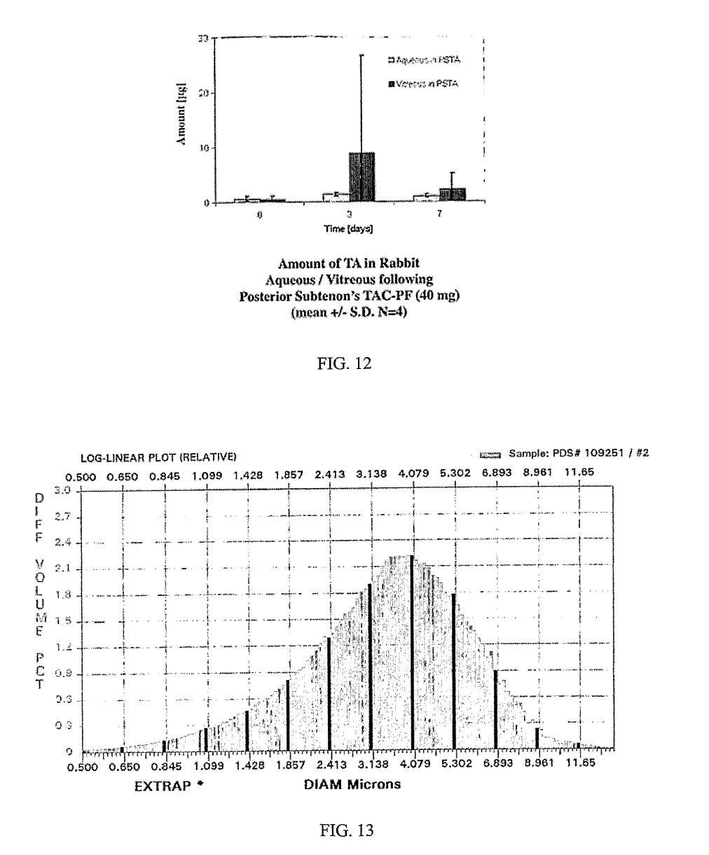

Rabbits were administered either 20 mg or 40 mg by injection at the post-anterior subtenon (FIG. 9, left bar, 20 mg dose of TAC-PF), anterior subtenon (FIG. 10 depicts aqueous and vitreous humor concentration after a 40 mg dose, and FIG. 11 depicts aqueous and vitreous humor concentration after a 20 mg dose), or posterior subtenon (FIG. 12 depicts aqueous and vitreous humor concentration after a 40 mg dose). In each transcleral administration of a 20 mg or 40 mg dose of the TAC-PF pharmaceutical compositions, triamcinolone acetonide is detected in the aqueous and vitreous humor of the treated eye at 0, 3, and 7 days post injection.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A pharmaceutical composition consisting essentially of:
(a) a therapeutically effective amount of a particulate steroid, wherein
(i) the steroid has an average particle size of from about 2.2 microns to about 10 microns, and
(ii) the steroid is sparingly soluble or substantially insoluble in water,
(b) an excipient selected from the group consisting of methylcellulose and hydroxy(C₁-C₈)alkylmethylcellulose;
(c) an pharmaceutically acceptable salt; and
(d) water, wherein the composition is substantially free of preservatives and non-polysaccharide polymers.

2. A pharmaceutical composition consisting essentially of:
(a) particulate triamcinolone acetonide;
(b) an excipient selected from the group consisting of methylcellulose and hydroxy(C₁-C₈)alkylmethylcellulose;
(c) an pharmaceutically acceptable salt; and
(d) water, wherein the composition is substantially free of preservatives and non-polysaccharide polymers.

3. A pharmaceutical composition consisting essentially of:
(a) particulate triamcinolone acetonide having an average particle size of from about 2.2 microns to about 10 microns;
(b) an excipient selected from the group consisting of methylcellulose and hydroxy(C₁-C₈)alkylmethylcellulose;
(c) an pharmaceutically acceptable salt; and
(d) water, wherein the composition is substantially free of preservatives and non-polysaccharide polymers.

4. The pharmaceutical composition of claim 2, consisting essentially of
(a) 1-25 mg of particulate triamcinolone acetonide per milliliter of composition;
(b) methylcellulose or hydroxypropylmethylcellulose at a concentration of about 0.2% (w/v) to about 5% (w/v);
(c) sodium chloride present at a concentration of 0.7% (w/v) to about 1.1 (w/v); and
(d) water, wherein the composition is substantially free of preservatives and non-polysaccharide polymeric materials.

5. The pharmaceutical composition of claim 2 consisting essentially of
(a) 2-20 mg of particulate triamcinolone acetonide per milliliter of composition;
(b) hydroxypropylmethylcellulose at a concentration of about 0.2% (w/v) to about 5% (w/v);
(c) sodium chloride present at a concentration of 0.7% (w/v) to about 1.1 (w/v); and
(d) water, wherein the composition is substantially free of preservatives and non-polysaccharide polymeric materials.

6. The pharmaceutical composition of claim 2, wherein the composition further consist essentially of one or more additional therapeutic agents.

7. The pharmaceutical composition of claim 2, wherein the particulate triamcinolone acetonide is in amorphous form, crystalline form, semi-crystalline form, semi-amorphous form, or a mixture thereof.

8. The pharmaceutical composition of claim 2, wherein the particulate triamcinolone acetonide is at least partially in crystalline form, and about 10% or less of the steroid is in amorphous form.

9. The pharmaceutical composition of claim 2, wherein about 20% or less of the triamcinolone acetonide particles have a particle size of greater than 10 microns.

10. The pharmaceutical composition of claim 2, wherein about 10% or less of the triamcinolone acetonide particles have a particle size of greater than 10 microns.

11. The pharmaceutical composition of claim 2, wherein about 5% or less of the triamcinolone acetonide particles have a particle size of greater than 10 microns.

12. The pharmaceutical composition of claim 2, wherein about 3% or less of the triamcinolone acetonide particles have a particle size of greater than 10 microns.

13. The pharmaceutical composition of claim 2, wherein the pharmaceutical composition is free of preservatives.

14. The pharmaceutical composition of claim 2, wherein the pharmaceutical composition is free of dispersion agents.

15. The pharmaceutical composition of claim 2, wherein the triamcinolone acetonide particles have an average particle size of between about 2.2 microns and 10 microns.

16. The pharmaceutical composition of claim 2, wherein the triamcinolone acetonide particles have an average particle size of between about 2.5 microns and about 7 microns.

17. The pharmaceutical composition of claim 16, wherein the triamcinolone acetonide particles have an average particle size of between about 3 microns and about 5 microns.

18. The pharmaceutical composition of claim 2, packaged in a single dose vial.

19. A method of treating an animal for a condition of the eye comprising administering the pharmaceutical composition of claim 2 in conjunction with photodynamic therapy.

20. The method of claim 19, wherein the photodynamic therapy employs verteporfin.

21. The method of claim 20, wherein the method is used to treat choroidal neovascularization.

22. A method of making the pharmaceutical composition of claim 2, the method comprising mechanically mixing the steroid in a solution of the excipient under aseptic conditions, wherein the steroid is not heated.

23. A method of treating an animal for a condition in need of steroid therapy, the method comprising administering a therapeutically effective amount of the pharmaceutical composition of claim 2 to the animal.

24. A method of treating an animal for a condition in need of triamcinolone therapy, the method comprising administering a therapeutically effective amount of the pharmaceutical composition of claim 2 to the animal.

25. The method of claim 23, wherein the condition is an ocular condition.

26. The method of claim 25, wherein the ocular condition is retinopathy, uveitis, choroidal or posterior segment neovascularization, macular degeneration, macular edema, retinal vein occlusion, surgically induced inflammation, endophthalmitis, scleritis, or episcleritis.

27. The method of claim 23, wherein the condition is dermatitis, eczema, an insect bite, asthma, clinical inflammation, lesions, ulcers, osteoarthritis, rheumatoid arthritis, bursitis, epicondylitis, keloids, psoriasis, endocrine disorders, lupus, rheumatic carditis, herpes zoster ophthalmicus, colitis, irritable bowel syndrome, ulcerative colitis, gastroenteritis, Crohn's disease, hemolytic anemia, leukemia, lymphoma, or rhinitis.

28. The method of claim 24, wherein the pharmaceutical composition is injected into the vitreal space.

29. The method of claim 21, wherein the pharmaceutical composition is administered transclerally.

30. The method of claim 29, wherein the pharmaceutical composition is administered into the sub-Tenon's space.

31. The method of claim 29, wherein the pharmaceutical composition is administered to the posterior sub-Tenon's space.

32. The method of claim 29, wherein the pharmaceutical composition is administered to the anterior sub-Tenon's space.

33. The method of claim 29, wherein the pharmaceutical composition is administered posterior juxtasclerally or subconjunctivally.

34. The method of claim 29, wherein the pharmaceutical composition is administered peribulbar or retrobulbar.

35. The method of claim 29, wherein between 1 mg and about 200 mg of triamcinolone acetonide is administered transclerally.

36. The method of claim 35, wherein between about 10 and about 100 mg of triamcinolone acetonide is administered transclerally.

37. The method of claim 29, wherein at least a portion of the steroid administered in the pharmaceutical composition is localized to the vitreous.

38. The method of claim 37, wherein at least about 0.1 μg of the triamcinolone acetonide is localized to the vitreous.

39. The method of claim 38, wherein between about 0.2 μg and about 10 μg of the triamcinolone acetonide is localized to the vitreous.

40. A method of treating an animal for a ocular condition, the method comprising administering to the animal transclerally a therapeutically effective amount of the pharmaceutical composition consisting essentially of:
(a) particulate triamcinolone acetonide;
(b) an excipient selected from the group consisting of methylcellulose and hydroxy(C₁-C₈)alkylmethylcellulose;
(c) an pharmaceutically acceptable salt; and
(d) water, wherein the composition is substantially free of preservatives and non-polysaccharide polymers.

41. The method of claim 40, wherein the pharmaceutical composition is administered into the sub-Tenon's space.

42. The method of claim 40, wherein the pharmaceutical composition is administered to the posterior sub-Tenon's space.

43. The method of claim 40, wherein the pharmaceutical composition is administered to the anterior sub-Tenon's space.

44. The method of claim 40, wherein the pharmaceutical composition is administered posterior juxtasclerally or subconjunctivally.

45. The method of claim 40, wherein the pharmaceutical composition is administered peribulbar or retrobulbar.

46. The method of claim 23, wherein the condition is pain.

47. The method of claim 46, wherein the pain is joint pain, back pain, or neck pain.

48. The method of claim 23, wherein the pharmaceutical compositions are administered to the musculoskeletal system.

49. The method of claim 23, wherein the pharmaceutical composition is administered epidurally.

50. The method of claim 23, wherein the pharmaceutical composition is administered to a mucous membrane.

51. The method of claim 23, wherein the pharmaceutical composition is administered around the spine, intrathecally, interlaminar, through the intervertbral foramen, to a facet joint, to a disc, intraarticular, or intrabursal.

52. The method of claim 23, wherein the animal is selected from domesticated animals, primates, and humans.

53. The method of claim 52, wherein the animal is selected from the group consisting of rat, cat, dog, pig, rabbit, horse, cow, elephant, primate, and human.

54. The method of claim 52, wherein the animal is a human.

55. A method of visualizing the vitreous of an eye, the method comprising administering the pharmaceutical composition of claim 2 to the eye.

56. The method of claim 51, wherein the method is used in preparation for pars plana vitrectomy, internal limiting membrane peeling, macula hole repair, or epiretinal membrane removal.

57-84. (canceled)

85. The pharmaceutical composition of claim 2 wherein the triamcinolone acetonide concentration in the composition is from 10 mg/ml to 450 mg/ml.

86. The pharmaceutical composition of claim 2 wherein the triamcinolone acetonide concentration in the composition is from 10 mg/ml to 200 mg/ml.

87. The pharmaceutical composition of claim 3 consisting essentially of:
(a) 1-25 mg of particulate triamcinolone acetonide per milliliter of composition;
(b) methylcellulose or hydroxypropylmethylcellulose at a concentration of about 0.2% (w/v) to about 5% (w/v);
(c) sodium chloride present at a concentration of 0.7% (w/v) to about 1.1 (w/v); and
(d) water, wherein the composition is substantially free of preservatives and non-polysaccharide polymeric materials.

88. The pharmaceutical composition of claim 3 consisting essentially of:
(a) 2-20 mg of particulate triamcinolone acetonide per milliliter of composition;
(b) hydroxypropylmethylcellulose at a concentration of about 0.2% (w/v) to about 5% (w/v);
(c) sodium chloride present at a concentration of 0.7% (w/v) to about 1.1 (w/v); and
(d) water, wherein the composition is substantially free of preservatives and non-polysaccharide polymeric materials.

89. The pharmaceutical composition of claim 2 consisting essentially of:
(a) 10-450 mg of particulate triamcinolone acetonide per milliliter of composition;
(b) methylcellulose or hydroxypropylmethylcellulose at a concentration of about 0.2% (w/v) to about 5% (w/v);
(c) sodium chloride present at a concentration of 0.7% (w/v) to about 1.1 (w/v); and
(d) water, wherein the composition is substantially free of preservatives and non-polysaccharide polymeric materials.

90. The pharmaceutical composition of claim 89 wherein the triamcinolone acetonide concentration in the composition is from 10 mg/ml to 200 mg/ml.

91. The pharmaceutical composition of claim 3 consisting essentially of:
(a) 10-450 mg of particulate triamcinolone acetonide per milliliter of composition;
(b) methylcellulose or hydroxypropylmethylcellulose at a concentration of about 0.2% (w/v) to about 5% (w/v);
(c) sodium chloride present at a concentration of 0.7% (w/v) to about 1.1 (w/v); and
(d) water, wherein the composition is substantially free of preservatives and non-polysaccharide polymeric materials.

92. The pharmaceutical composition of claim 91 wherein the triamcinolone acetonide concentration in the composition is from 10 mg/ml to 200 mg/ml.

93. A pharmaceutical composition comprising:
(a) triamcinolone acetonide in a concentration of from 10 mg/ml to 450 mg/ml;
(b) an excipient selected from the group consisting of a methylcellulose, a hydroxy(C₁-C₈)alkylmethylcellulose, Carbomer 940, polyethylene glycol, and polyvinyl alcohol; and
(c) an aqueous carrier; wherein the pharmaceutical composition is free of classical preservatives.

94. The pharmaceutical composition of claim 3 wherein the triamcinolone acetonide concentration in the composition is from 10 mg/ml to 200 mg/ml

95. The pharmaceutical composition of claim 93 wherein the composition is free of dispersion agents.

96. The pharmaceutical composition of claim 93 wherein the triamcinolone acetonide has an average particle size of from 2.2 microns to 10 microns.

97. The pharmaceutical composition of claim 93 consisting essentially of the triamcinolone acetonide in a concentration of from 10 mg/ml to 450 mg/ml; the excipient; and the aqueous carrier.

98. The pharmaceutical composition of claim 95 consisting essentially of the triamcinolone acetonide in a concentration of from 10 mg/ml to 450 mg/ml; the excipient; and the aqueous carrier.
